(19) United States
(12) Patent Application Publication
(10) Pub. No.: US 2004/0260193 A1
(43) Pub. Date: Dec. 23, 2004

(54) Cardiac impulse detector

(76) Inventor: Anthony F. LaSala, West Hartford, CT (US)

Correspondence Address: MCCARTER & ENGLISH LLP, CITYPLACE I, 185 ASYLUM STREET, HARTFORD, CT, 06103, US

(21) Appl. No.: 10/830,559

(22) Filed: Apr. 23, 2004

Related U.S. Application Data
(63) Continuation-in-part of application No. 09/570,695, filed on May 12, 2000, now Pat. No. 6,726,635.

(51) Int. Cl. A61B 5/02

(52) U.S. Cl. 600/528

(57) ABSTRACT

An apparatus and method for detecting infrasonic cardiac apical impulses of a patient including a sensor disposable in contact with skin of the patient for producing a signal responsive to a motion of the skin at an infrasonic cardiac apical impulse point of the patient. A first circuit coupled to the first sensor for generating at least one audible output in response to the first signal and indicative of the infrasonic cardiac apical impulse.

CROSS-REFERENCE TO RELATED APPLICATIONS

This patent application is a Continuation-In-Part (CIP) Application of U.S. patent application Ser. No. 09/570,695 filed May 12, 2000, co-pending herewith, the entire disclosure of which is hereby incorporated by reference.

### FIELD OF THE INVENTION

The present invention relates to medical diagnostic instruments, and more particularly, to such instruments for detecting abnormal heart functions.

### BACKGROUND INFORMATION

Patients occasionally develop heart disease, the prompt and timely discovery of which can be determinative of patients' health and survival. Until the 19th century, medical caregivers had to press their ears against patients' chests in order to hear heart sounds. When the stethoscope (“spy of the chest” in Greek) was introduced by René Laennec (1781-1826), it enabled medical caregivers to hear heart sounds with improved ease and clarity. “In search of the perfect stethoscope that hears all heart sounds, and explains them to you.” (Laennec)

The ballistic recoiling of the heart produces a vibration when it moves its apex upward, rightward, and against the underside of the chest wall before the ejection of blood. This motion or vibration is typically inaudible and infrasonic, having a sound frequency of less than about 30 Hertz. There are other low frequency, low amplitude vibrations which normally occur during cardiac filling. There are also abnormal cardiac vibrations with sound frequencies as low as about 10 Hertz, but of high amplitude that occur when the heart fills abnormally. The period of cardiac filling is called diastole, and when these abnormal vibrations occur, they indicate diastolic dysfunction of the heart. These abnormal vibrations during diastole are called pathologic gallops. Some gallops are faint and difficult to hear, and some are infrasonic.

There are two types of pathologic gallops of primary clinical significance: A S4 type of gallop, which occurs during late diastole; and a S3 type of gallop, which occurs during early diastole. Many gallops are palpable and visible even when they are inaudible. This is because they are of high-energy amplitude despite their low frequencies. Detection of gallops is very important, and can lead to the diagnosis and treatment of such cardiac disorders as hypertrophic heart syndromes, valvular lesions, cardiomyopathies, and congenital heart problems.

A visual and palpable assessment of cardiac motion of a patient in the supine position may be made at the left chest wall near the left breast. This location is called the cardiac apical impulse, and for purposes of clarity is also herein referred to as the cardiac apical impulse point. The cardiac apical impulse point is a single area typically less than about 15 millimeters in diameter. The skin motion at this location is normally caused by the recoiling of the heart when it moves its ventricular apex upward, rightward, and against the underside of the chest wall. Presently, medical caregivers may examine the heart motions at the cardiac apical impulse point by placing their fingertips against the skin at this point to enable tactile detection of apical impulses having sufficient amplitude.

Over the past 60 years, sophisticated and elaborate laboratory apparatus have been developed to detect and record heart movements, and enable medical caregivers to analyze the data for indications of abnormal heart conditions. The apexcardiogram (“ACG”), for example, which was in popular use until the early 1980's, was capable of revealing low frequency heart motions by means of electromechanical sensors affixed to a patient's chest. The ACG signals were recorded on a strip chart recorder for later analysis. An electrocardiogram (“EKG”) and a separate phonocardiogram were required to be performed contemporaneous with the ACG in order to provide correlation between the low frequency heart motions and the additional heart signals. The three charts were then correlated, as by technicians, for later analysis by caregivers. Although this method was very useful for detecting heart irregularities in suspected cases, the time delay incurred by a patient between seeing a physician for referral to an ACG laboratory, testing in the laboratory by technicians, correlation of strip chart results, and analysis and diagnosis by at least one physician, generally hindered prompt and effective treatment in time-critical cases. In addition, the large expense for this labor-intensive procedure may have precluded its use in many instances.

By the mid-1980's, the ACG had been generally displaced by the echocardiogram. The echocardiogram uses ultrasonic waves to monitor heart function and provides more detail than the ACG. Unfortunately, the echocardiogram suffers from some of the same drawbacks as the ACG, including the requirement for special laboratory testing and associated expense. Like the ACG, the echocardiogram also fails to produce recognizable sounds indicative of the infrasonic heart motions, and therefore fails to disclose a method for their discovery.

Various other prior art systems are also directed toward monitoring human heart function. For example, U.S. Pat. No. 5,218,969 to Bredesen et al. (“the '969 patent”) depicts an electronically enhanced stethoscope for detecting heart sounds. However, the '969 patent teaches filtering out sounds below 50 Hz (see FIG. 3F). Since human hearing is generally recognized to extend to at least as low as 30 Hz, the stethoscope of the '969 patent is not capable of detecting heart vibrations of frequency below the range of human hearing, even if it may amplify low amplitude sounds which are above 50 Hz. Accordingly, the electronic stethoscope of the '969 patent does not detect infrasonic cardiac apical impulses, and in fact is incapable of detecting any phenomena emitting a frequency below 50 Hz.

U.S. Pat. No. 5,178,151 to Sackner (“the '151 patent”) shows another system for detection of heart irregularities. The '151 patent shows placement of a plurality of motion transducers about the thoracic region of a patient's chest wall. Blood vessel volume, blood pressure waveforms, and other thoracic motions including respiratory and cardiac apical motions are measured as conglomerate signals that must be further analyzed to determine the presence of heart irregularities. Due in part to its bulk, complexity, cost, and requirement for further analysis, this system suffers from design constraints that generally preclude its inclusion in a general caregiver's office. The apparatus of the '151 patent further lacks provision for transmitting the acoustic heart waveform data typically relied on during a routine physical examination.

The basic acoustic stethoscope, whether electronically amplified, filtered or not, can only be used to hear what Rene Laennec heard with his original wooden device. Only a small percentage of the vibrations of the heart are actually detected by an acoustic stethoscope. These audible vibrations range between about 40 Hertz to 500 Hertz and about 0.002 to 0.5 dynes/cm² (amplitude). The remaining vibrations are inaudible because of the typical thresholds of human hearing.

Infrasonic vibrations of sufficient amplitude have heretofore only been detectable with bulky, complex, and costly apparatus requiring labor intensive analysis. Heart gallops rest near the division of audible and infrasonic vibrations. Heart gallops have been called the heart's “cries for help.” Detection of these vibrations is important in diagnosing cardiac pathology and is why palpation of the cardiac apical impulse is an extremely important, yet often neglected, part of the cardiac exam.

All in all, the above-described prior art fails to recognize the utility of detecting infrasonic heart motions and producing outputs that are indicative of those motions. Such prior art also fails to put infrasonic heart motion data in context with traditional acoustic heart data. It is therefore an object of the present invention to overcome the above-described drawbacks and disadvantages of the prior art.

### SUMMARY OF THE INVENTION

The present invention is directed to a cardiac impulse detector and related method for use in routine cardiac examinations, which employs a sensor capable of detecting infrasonic cardiac apical impulses of a patient. The detector produces audible and optionally visual outputs indicative of those impulses for contemporaneous consideration by a medical caregiver when a flexible substrate of the sensor is placed in contact with the patient's skin surface at the cardiac apical impulse point.

In one embodiment, a sensing protrusion or button is placed in contact with the skin surface of the patient at the patient's cardiac apical impulse point. The cardiac apical impulse point is located near the left breast. The sensing button is mounted to a piezoelectric sensor, and causes the sensor to respond to the infrasonic heart motions or impulses at the cardiac apical impulse point of the patient. A circuit is electronically connected to the piezoelectric sensor and generates audible and visual outputs indicative of the heart motions. The piezoelectric sensor is housed in one end of an hourglass shaped housing, which provides the caregiver with a convenient grip for holding the device against the cardiac apical impulse point.

This embodiment of the detector further employs a traditional acoustic diaphragm mounted at the opposite end of the housing relative to the piezoelectric sensor. The acoustic diaphragm can transmit acoustic heart sounds to an earpiece worn by the caregiver when the acoustic end of the sensor housing is placed in contact with the patient's chest, and a selection manifold has been rotated 180 degrees in order to transmit the traditional acoustic sounds instead of the signals indicative of infrasonic heart motions. The sounds may be electronically amplified and/or filtered. This embodiment has the distinct advantage of placing the audible signal indicative of an infrasonic cardiac impulse in temporal context with the traditional acoustic cardiac sounds familiar to the caregiver.

In accordance with another aspect of the present invention, an apparatus is provided for detecting infrasonic cardiac apical impulses of a patient. The apparatus comprises a flexible substrate including (i) a skin-contacting surface located on one side of the substrate that is disposable in contact with a skin surface region of a patient defining an infrasonic cardiac apical impulse point, and is movable with the contacted skin surface region in response to a subaudible motion of the contacted skin at the infrasonic cardiac apical impulse point; and (ii) a reflective surface located on an opposite side of the substrate relative to the skin-contacting surface and movable with the skin-contacting surface in response to a subaudible motion of the contacted skin at the infrasonic cardiac apical impulse point. A light source, such as a laser, is spaced apart from and faces the reflective surface of the substrate. The light source transmits light onto the reflective surface, and the reflective surface reflects light transmitted thereon by the light source. An optical sensor is spaced apart from and faces the reflective surface. The optical sensor receives reflected light directed by the reflective surface and generates a first signal indicative of movement of the reflective and skin-contacting surfaces and corresponding to a subaudible motion of the contacted skin at the infrasonic cardiac apical impulse point. An electric circuit is coupled to the optical sensor for generating (i) an audible output and/or (ii) a visual output, in response to the first signal and indicative of an infrasonic cardiac apical impulse.

In accordance with another aspect, the present invention is directed to a method for detecting infrasonic cardiac apical impulses of a patient, comprising the following steps:

(i) providing a flexible substrate including a skin-contacting surface located on one side of the substrate and a reflective surface located on an opposite side of the flexible substrate relative to the skin-contacting surface;

(ii) positioning the skin-contacting surface of the flexible substrate in contact with a skin surface region of the patient defining an infrasonic cardiac apical impulse point on the patient's chest;

(iii) allowing movement of the skin-contacting and reflective surfaces of the flexible substrate with movement of the skin surface region of the patient in response to a subaudible motion of the skin at the infrasonic cardiac apical impulse point;

(iv) transmitting light from a light source onto the reflective surface of the flexible substrate positioned on the skin surface region of the patient defining the infrasonic cardiac apical impulse point;

(v) reflecting transmitted light from the light source with the reflective surface of the flexible substrate positioned on the skin surface region of the patient defining the infrasonic cardiac apical impulse point;

(vi) receiving with an optical sensor reflected light directed by the reflective surface, and generating a first signal indicative of movement of the reflective and skin-contacting surfaces and corresponding to a subaudible motion of the skin at the infrasonic cardiac apical impulse point;

(vii) processing the first signal electronically; and

(viii) generating (i) an audible output and/or (ii) a visual output, indicative of an infrasonic cardiac apical impulse.

In accordance with another aspect, the present invention is directed to a flexible substrate engageable with a patient's skin for detecting infrasonic cardiac apical impulses. The flexible substrate is operable in connection with an apparatus including a light source spaced apart from and facing the substrate to transmit light thereon; an optical sensor spaced apart from and facing the substrate for receiving reflected light from the substrate and generating a signal indicative of movement of the substrate and corresponding to a subaudible motion of the contacted skin at an infrasonic cardiac apical impulse point; and a circuit coupled to the optical sensor for generating at least one of (i) an audible output and (ii) a visual output, in response to the signal and indicative of an infrasonic cardiac apical impulse. The flexible substrate comprises (i) a flexible skin-contacting surface located on one side of the substrate that is disposable in contact with a skin surface region of a patient defining an infrasonic cardiac apical impulse point, and is movable with the contacted skin surface region in response to a subaudible motion of the contacted skin at the infrasonic cardiac apical impulse point; and (ii) a flexible reflective surface located on an opposite side of the substrate relative to the skin-contacting surface and movable with the skin-contacting surface in response to a subaudible motion of the contacted skin at the infrasonic cardiac apical impulse point for reflecting light transmitted thereon by the light source onto the optical sensor.

In one such embodiment, the flexible substrate is in the form of an adhesive-backed reflective patch that is used in cooperation with a sensor assembly and a circuit assembly to detect the cardiac apical impulse of a patient. In this embodiment, the sensor assembly has means for emitting and/or detecting light and the circuit assembly has means suitable for manipulating data from the sensing assembly to generate signals indicative of movement of the reflective patch placed in direct contact with a surface of the patient at the cardiac apical impulse point. The signals generated by movement of the reflective patch in response to cardiac impulses can be audibly and/or visually indicated to provide a caregiver with means to audibly and/or visually observe and correlate normal and abnormal infrasonic cardiac apical impulses for cost-effective and timely diagnosis of pending heart failure or other conditions.

In accordance with another aspect, the present invention is directed to a method for detecting infrasonic cardiac apical impulses of a patient. The method comprises the following steps:

(1) providing a flexible substrate including a flexible skin-contacting surface located on one side of the substrate and a flexible reflective surface located on an opposite side of the flexible substrate relative to the skin-contacting surface;

(2) positioning the skin-contacting surface of the flexible substrate in contact with a skin surface region of the patient defining an infrasonic cardiac apical impulse point on the patient's chest;

(3) allowing movement of the skin-contacting and reflective surfaces of the flexible substrate with movement of the skin surface region of the patient in response to a subaudible motion of the skin at the infrasonic cardiac apical impulse point;

(4) transmitting light from a light source onto the reflective surface of the flexible substrate positioned on the skin surface region of the patient defining the infrasonic cardiac apical impulse point;

(5) reflecting transmitted light from the light source with the reflective surface of the flexible substrate positioned on the skin surface region of the patient defining the infrasonic cardiac apical impulse point;

(6) receiving with an optical sensor reflected light directed by the reflective surface and generating a first signal indicative of movement of the reflective and skin-contacting surfaces and corresponding to a subaudible motion of the skin at the infrasonic cardiac apical impulse point;

(7) processing the first signal electronically, and generating at least one of (i) an audible output, and (ii) a visual output, indicative of an infrasonic cardiac apical impulse.

In one embodiment, the light source and optical sensor are mounted remotely from the flexible substrate and patient. In another such embodiment, the light source and optical sensor are fixedly mounted relative to the flexible substrate. Preferably, the patient is seated in an upright position to bring the apex of the heart into close proximity with, and preferably in contact with, the chest wall to facilitate transmitting the kinetic energy of apical impulses into the chest wall, and in turn, facilitate movement of the skin contacted by the flexible substrate in response to the apical impulses.

One advantage of the present invention is that it may provide an efficient way to screen patients for abnormal infrasonic vibrations or pathological gallops during routine physical examinations, a clearly desirable improvement over current procedure which requires elaborate set-up of bulky apparatus. Other objects and advantages of the present invention will become apparent in view of the following Detailed Description of the Preferred Embodiments and accompanying drawings.

### BRIEF DESCRIPTION OF THE DRAWINGS

FIG. 1 is a somewhat schematic, elevational view of a first embodiment of a cardiac impulse detector of the present disclosure.

FIG. 2 is a cross-sectional view of the cardiac impulse detector taken along line 2-2 of FIG. 1.

FIG. 3 is an exploded, partial, somewhat schematic, cross-sectional view of the cardiac impulse detector of FIGS. 1 and 2.

FIG. 4 is a schematic illustration of electronic circuitry of the cardiac impulse detector of FIGS. 1, 2 and 3, for driving a speaker and Light Emitting Diode in response to a sensed heart motion at the cardiac apical impulse point.

FIG. 5 is a somewhat schematic, elevational view of a second embodiment of a cardiac impulse detector embodying the present invention.

FIG. 6 is a cross-sectional view of the cardiac impulse detector taken along line 6-6 of FIG. 5.

FIG. 7 is a schematic illustration of electronic circuitry of the cardiac impulse detector of FIGS. 5 and 6, for driving a speaker in response to a sensed heart motion at the cardiac apical impulse point.

FIG. 8 is a cross-sectional view of a third embodiment of a cardiac impulse detector embodying the present invention.

FIG. 9 is a schematic illustration of a fourth embodiment of a cardiac impulse detector embodying the present invention.

FIG. 10a is an exploded perspective view of an adhesive reflective patch in accordance with an illustrative aspect of the cardiac impulse detector of FIG. 9.

FIG. 10b is a plan view of a number of adhesive reflective patches in association with a common releasable backing in accordance with an illustrative aspect of the cardiac impulse detector of FIG. 9;

FIG. 10c is a perspective view of a number of adhesive reflective patches in association with a common releasable backing in accordance with another illustrative aspect of the cardiac impulse detector of FIG. 9;

FIG. 11 is a first cross-sectional view of a sensor assembly in accordance with an illustrative aspect of the cardiac impulse detector of FIG. 9.

FIG. 12 is a second cross-sectional view of the sensor assembly of FIG. 11.

FIG. 13 is a schematic illustration of electronic circuitry of the cardiac impulse detector of FIG. 9 for driving a speaker and/or a light source in response to a sensed heart motion at the cardiac apical impulse point.

FIGS. 14-16 are graphical representations of a various exemplary cardiac pathologies.

FIG. 17 is a schematic diagram of an exemplary cardiac impulse detector system in accordance with the present invention.

FIG. 18 is a part front view of a person's torso showing various points for obtaining optimal impulse signals.

FIG. 19 is a schematic diagram illustrating an optimal position and posture for obtaining optimal impulse signals.

FIG. 20 is a part front view of a person's torso highlighting the left ventricle of the heart as a preferred point for obtaining optimal impulse signals.

### DETAILED DESCRIPTION OF THE PREFERRED EMBODIMENTS

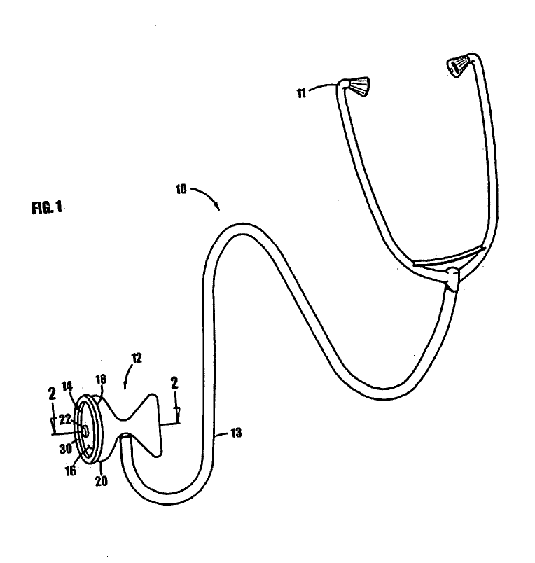

In FIG. 1, a first embodiment of the cardiac impulse detector of the present disclosure is indicated generally by the reference numeral 10. The cardiac impulse detector 10 comprises an earpiece 11 connected to a sensor assembly 12 via acoustic tubing 13. The sensor assembly 12 comprises a diaphragm 14 including a piezoelectric element 16 superimposed over a substrate 18, wherein the diaphragm is mounted around its circumference to a housing 20. A sensing protuberance 22 is mounted to the piezoelectric element 16. A dampening member 30 is mounted to the housing 20 at one end, and surrounds the sensing protuberance 22 without contact. The housing 20 is hourglass shaped in order to transmit acoustic pressure waves with minimal attenuation and distortion, and to provide a convenient grip for placement of a caregiver's hand.

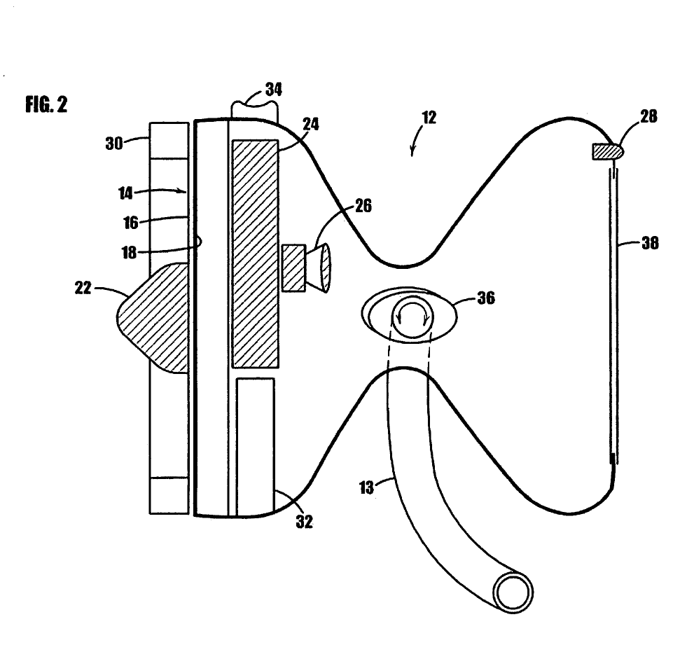

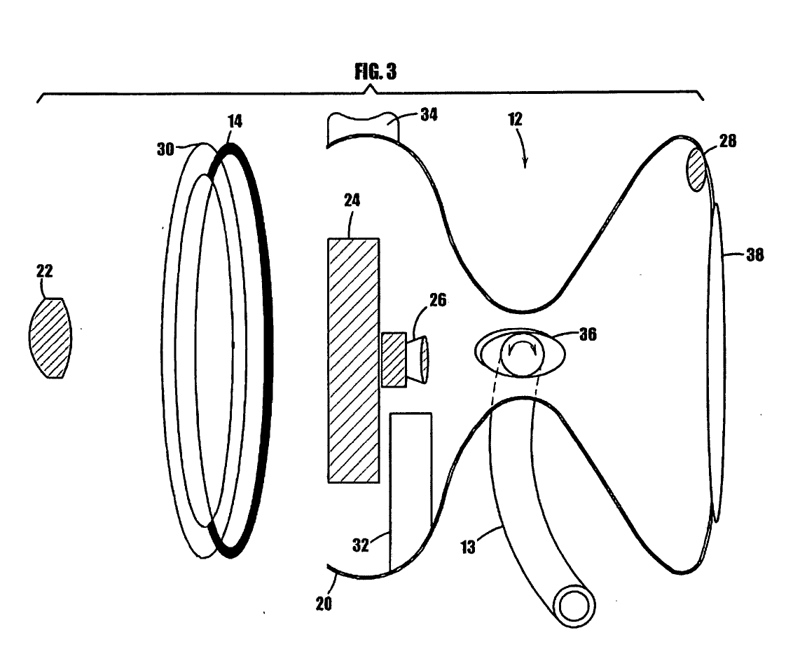

Turning to FIGS. 2 and 3, the sensing protuberance 22 is mounted to the piezoelectric element 16 at about its center. In this embodiment, the sensing protuberance 22 is made of nylon and is rounded and convex, allowing it to nest longitudinally in the intercostal space at a patient's cardiac apical impulse point, thus coming into contact with the skin tissue over the cardiac apex. As the ventricular apex of the heart strikes the internal surface of the rib cage, it generates an impulse, which, coupled through the tissue, strikes the sensing protuberance 22. As may be recognized by those skilled in the pertinent art based on the teachings herein, the sensing protuberance 22 may take any of numerous different shapes for performing the functions of the exemplary protuberance, such as a globular, ovate, or other substantially smooth shape. Likewise, the sensing protuberance 22 may be made of any of numerous different materials for performing the functions of the protuberance described herein, such as polyurethane or thermoplastic rubber.

When the sensing protuberance 22 is struck, it deflects the diaphragm 14, and thus the piezoelectric element 16. The piezoelectric element 16 is a natural mechanical differentiator and a transducer of mechanical movement into electrical signals. When deflected, it generates a momentary charge. It is not a sustained voltage potential but a voltage spike that decays rapidly, indicating proportional changes in forces applied to the diaphragm 14. This characteristic is advantageous for this application in order to detect low frequency or infrasonic cardiac movements. When the cardiac apex strikes the sensing protuberance 22, the momentary charge is generated by the piezoelectric element 16. An electronic circuit 24 is connected to the piezoelectric element 16, and electronically detects the momentary charge indicative of the heart motion at the cardiac apical impulse point. An audio speaker 26 and a light emitting diode (“LED”) 28 are connected to the electronic circuit 24, and generate outputs corresponding to the detected cardiac apical motion. These indications provide the caregiver the means to audibly and visibly observe and correlate normal and abnormal infrasonic cardiac apical impulses. As may be recognized by those skilled in the pertinent art based on the teachings herein, the speaker 26 may be supplemented or replaced by any of numerous different audible transducers for performing the functions of the speaker described herein, such as a piezoelectric buzzer, or other audible indicator. Likewise, the LED 28 may be supplemented or replaced by any of numerous different visible indicators for performing the functions of the LED described herein, such as an electrical light bulb, liquid crystal display, graphical monitor or computational device.

The diaphragm's substrate 18 is made of MYLAR® film, mounted at its outer diameter to the housing 20. As may be recognized by those skilled in the pertinent art based on the teachings herein, the MYLAR® film may be replaced with any of a number of suitable materials for performing the functions of the substrate described herein, such as spring steel or other resilient material. The diaphragm 14 is preferably taut so that it is mechanically biased, which will, in turn, lead to enhanced sensitivity when the protuberance 22 is placed on a patient.

The location of the dampening member 30 around the outer diameter of the diaphragm 14 provides a mechanical stabilizing and decoupling effect when the cardiac impulse detector 10 is placed on a patient's skin. In this embodiment, the dampening member 30 is in the form of a ring and made of foam rubber. As may be recognized by those skilled in the pertinent art based on the teachings herein, the dampening member 30 may comprise any of numerous different materials or mechanisms which now or later become known for performing the functions of the dampening member described herein, such as foam, rubber, soft polyurethane, or a hydraulic fluid damper. As also may be recognized by those skilled in the pertinent art based on the teachings herein, the dampening member 30 may take any of numerous different shapes for performing the functions of the dampening member described herein, such as oval, elongated, or rectangular. This configuration for interfacing the diaphragm 14 to the cardiac apex promotes improved signal acquisition and reduced secondary motion from the caregiver's hand or inadvertent patient movements.

The cardiac impulse detector 10 further comprises a battery 32 mounted to the housing 20, and electrically connected to a momentary power switch 34, which, in turn, is electrically connected to the electronic circuit 24 to supply power for the circuit and for the indicators 26 and 28. The battery 32 is to be replaced if the indicators do not activate when the sensing protuberance 22 is intentionally touched. In this embodiment, the detector 10 is only active when the momentary power switch 34 is depressed, typically by operation of a caregiver's finger, which is intended to be done while the detector 10 is in place on a patient. When the finger moves from the switch 34, the power goes off, conserving energy in the battery 32.

The cardiac impulse detector 10 further comprises a traditional acoustic diaphragm 38 mounted to the opposite side of the housing 20 relative to the diaphragm 14. In operation, the sensor housing 20 is preferably placed on a patient's chest with the acoustic diaphragm 38 in unobstructed contact with the patient's chest in order to obtain the least amount of acoustic attenuation. The acoustic diaphragm responds to local sound-pressure waves in the tissue medium against its outer surface by reproducing the sound-pressure waves in the gaseous medium against its inner surface. Thereafter, the waves are propagated in substantially unattenuated form towards the closest pressure equilibrium point, normally an earpiece. The acoustic diaphragm effectively amplifies incident sounds by receiving sound pressure over a larger area than that of the equilibrium point or earpiece. As may be recognized by those skilled in the pertinent art based on the teachings herein, the traditional acoustic diaphragm 38 may be augmented or replaced with any of numerous different sensors for performing the functions of the exemplary acoustic diaphragm, such as an electronic microphone or similar device for sensing acoustic heart sounds. For example, a piezoelectric microphone may be employed to sense acoustic heart sounds, while an electronic amplifier and speaker transduce the electronic signal back into audible sound.

A selection manifold 36 is acoustically coupled to the speaker 26 and the acoustic diaphragm 38. The selection manifold enables alternate acoustic connection of the speaker 26 and the diaphragm 38. As shown in FIG. 1, the earpiece 11 is connected through the acoustic tubing 13 to the selection manifold 36 to receive sound from at least one of the speaker 26 and the acoustic diaphragm 38. A caregiver may rotate the selection manifold 36 one half turn (180°) to create a path for one sound source and block the other, thereby blocking loss of the desired sound-pressure across the unused diaphragm. The manifold 36 is turned in the opposite direction to switch between the sound sources 38 and 26.

Turning now to FIG. 4, the electronic circuit 24 employs a front-end charge amplifier sub-circuit 40 of typically high gain to amplify the voltage signal generated by the piezoelectric element 16. A first differential output of the piezoelectric element 16 is connected through a resistor 62 to an inverting input of an operational amplifier (“op-amp”) 58. The inverting input of the op-amp 58 is also connected to a negative feedback path comprising a capacitor 42 and a resistor 60, connected in parallel between the output of the op-amp 58 and its inverting input. The non-inverting input of the op-amp 58 is connected to a reference potential V_{REF}, prescribing the gain. A second differential output of the piezoelectric element 16 is connected through a resistor 63 to an inverting input of an op-amp 59. The inverting input of the op-amp 59 is also connected to a negative feedback path comprising a capacitor 43 and a resistor 61, connected in parallel between the output of the op-amp 59 and its inverting input. The non-inverting input of the op-amp 59 is connected to the reference potential V_{REF}, prescribing the gain. The capacitors 42 and 43 in the feedback paths enhance the amplification of the voltage potential generated by the piezoelectric element 16. A secondary amplification stage is implemented by an op-amp 64, which receives at its non-inverting input the voltage from the op-amp 59 divided across a resistor 65, with a resistor 66 completing the path to ground potential. The op-amp 64 receives at its inverting input the sum of the output of the op-amp 58 received across a resistor 68, and the direct feedback signal received across a resistor 70.

After the amplification stage, a comparator sub-circuit 45 generates a voltage pulse of variable duration corresponding to the detected cardiac apical impulse. The output from the op-amp 64 is connected across a capacitor 44 to the inverting input of a variable threshold comparator 52. The capacitor 44 functions as an AC coupled filter. The inverting input of the comparator 52 is also connected to the anode of a diode 48. Each cardiac apical impulse charges a threshold capacitor 46 through the diode 48. The cathode of the diode 48 is connected to the non-inverting input of the comparator 52. The capacitor 46 is connected to ground in parallel with a resistor 50, which sets a typically slow time constant. The resistor 50 drains charge from the threshold capacitor 46, tending to keep its voltage within the operating range of the comparator 52. The diode 48 creates a voltage difference between the two inputs to the comparator 52 by allowing current to pass from the inverting input towards the non-inverting input, thus incurring a typical voltage drop. The cathode of the diode 48 is connected to the parallel combination of the capacitor 46 and the resistor 50 to ground potential, as well as to the non-inverting input of the comparator 52. When the input signal from the capacitor 44 is below the voltage level of the threshold capacitor 46, which occurs between apical impulses, the output of the comparator 52 relative to ground potential is about one half of battery voltage or V+. When there is a cardiac apical impulse, the voltage level of the input signal from the capacitor 44 rises above the voltage level of threshold capacitor 46, switching the output to about negative one half of battery voltage or V−, thus creating a negative voltage pulse. The initial pulse-width or duration will be the width of the pulse generated by the piezoelectric diaphragm 16.

Typically, the pulse produced by the comparator 52 lacks sufficient duration to activate the speaker 26 or other audible indicator, or the LED 28 or other visual indicator. An output driver sub-circuit 56 utilizes a one-shot integrated circuit (“one-shot”) 54 that is triggered by the pulse from comparator 52 to generate a single pulse of substantially constant duration. The duration of the generated pulse is independent of the duration of the incoming pulse from the comparator 52. The duration of the pulse generated by the one-shot 54 can be varied in order to optimize the outputs of the indicators 26 and 28, and to minimize the current required from the battery 32 of FIGS. 2 and 3. The output of the one-shot 54 is connected to a resistor 72, which, in turn, is connected to the base of a NPN BJT transistor 74. An emitter of the transistor 74 is connected to ground potential, and a collector of the transistor 74 is connected to the speaker 26, which, in turn, is connected to V+. Consequently, the transistor 74 drives the speaker 26 in response to the cardiac apical impulse sensed by the piezoelectric element 16. As may be recognized by those skilled in the pertinent art based on the teachings herein, the exemplary NPN BJT transistor 74 may be replaced with any of numerous different switching devices for performing the functions of the exemplary transistor, such as a FET or other suitable switching device for driving the speaker 26.

The output of the one-shot 54 is also connected to a resistor 76, which, in turn, is connected to the base of a transistor 78. An emitter of the transistor 78 is connected to ground potential, and a collector of the transistor 78 is connected to LED 28, which, in turn, is connected to V+ through a resistor 80. As may be recognized by those skilled in the pertinent art based on the teachings herein, the exemplary NPN BJT transistor 78 may be replaced with any of numerous different switching devices for performing the functions of the exemplary transistor, such as a FET or other suitable switching device for driving the LED 28.

The LED 28 can be seen by the caregiver as the cardiac impulse detector is held in place on a patient. The audible signal, generated by the speaker 26, is connected through the tubing 13 of FIG. 1 to the earpiece 11 and can be heard by the caregiver as the cardiac impulse detector is held in place on a patient.

In operation, the sensor assembly 12 of the cardiac impulse detector 10 is placed on the cardiac apical impulse point of a patient, with the sensing protuberance 22 contacting the patient's skin surface at the cardiac apical impulse point. A cardiac apical impulse induces motion of the sensing protuberance 22, which in turn causes motion of the piezoelectric element 16. The circuit 24 of FIG. 2 processes the signal from the piezoelectric element 16, and drives the speaker 26 and the LED 28 in response to the cardiac apical impulse. When a single gallop is present, the speaker will sound twice per heartbeat in a repeating series, and the LED will correspondingly flash twice in a repeating series. At a typical resting pulse rate of about 60 heartbeats per minute, the period between the two sounds indicative of the gallop is noticeably shorter than the period between successive heartbeats. If, on the other hand, the cardiac apical impulse is normal, then the speaker and LED will signal just once in a repeating series, at a rate equal to the pulse rate and will be timed with the upstroke of a peripheral pulse. Abnormal cardiac apical impulses will cause a repeating series of indications representative of the actual number of local peaks in the cardiac apical impulse waveform, where a single gallop waveform has two such peaks, and two gallops have three peaks. The housing 20 may be positioned adjacent to a patient's chest surface so that the acoustic diaphragm 38 picks up acoustic cardiac sounds and transmits them to the selection manifold 36. The selection manifold 36 may be rotated to selectively transmit the sound generated by at least one of the speaker 26 and the acoustic diaphragm 38 by rotation of the manifold. The earpiece 11 receives the acoustic signal transmitted by the selection manifold. As may be recognized by those skilled in the pertinent art based on the teachings herein, an electronic amplifier or similar device which is currently or later becomes known for performing the functions of an amplifier may be connected to the earpiece to enhance the quality of the sound received by the caregiver.

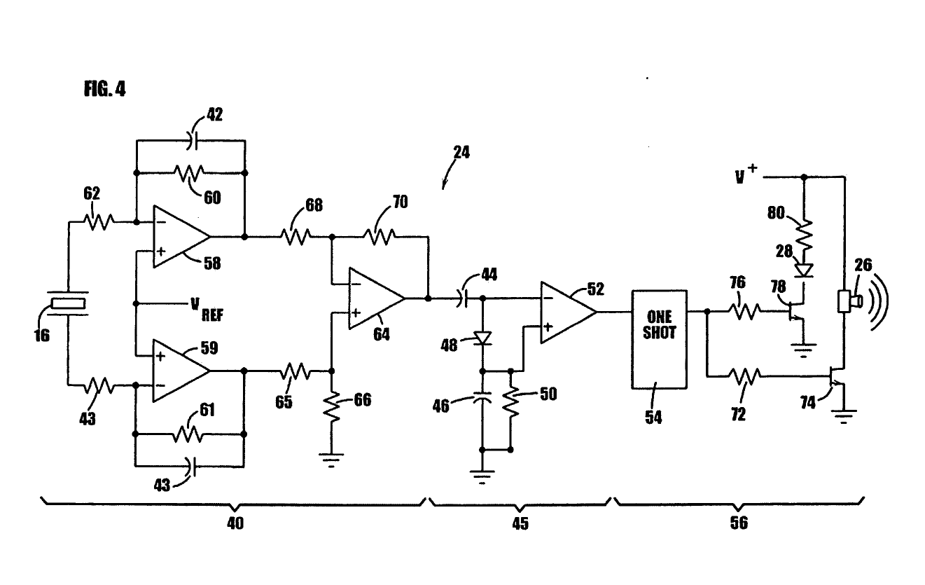

In FIGS. 5 through 7, a second embodiment of the cardiac impulse detector of the present invention is indicated generally by the reference numeral 110. The cardiac impulse detector 110 is substantially similar to the cardiac impulse detector 10 described above, and therefore like reference numerals preceded by the numeral 1 are used to indicate like elements. The cardiac impulse detector 110 of FIG. 5 differs from that of FIG. 1 primarily in that the sensor assembly 112 utilizes a laser diode and compatible sensing scheme in lieu of the piezoelectric sensing scheme of the sensor assembly 12. The application and overall operation of the cardiac impulse detector 110 is substantially similar to that of the cardiac impulse detector 10. As shown in FIG. 5, the cardiac impulse detector 110 comprises an earpiece 111 connected to a sensor assembly 112 via acoustic tubing 113. The sensor assembly 112 comprises a flexible diaphragm 114 mounted about its circumference to a housing 120. An electrical power switch 134 is mounted to the housing 120 for activating the cardiac impulse detector. An audio amplifier 115 is connected to the tubing 113, and is electrically connected to the power switch 134, for enhancing the quality of the audio signal.

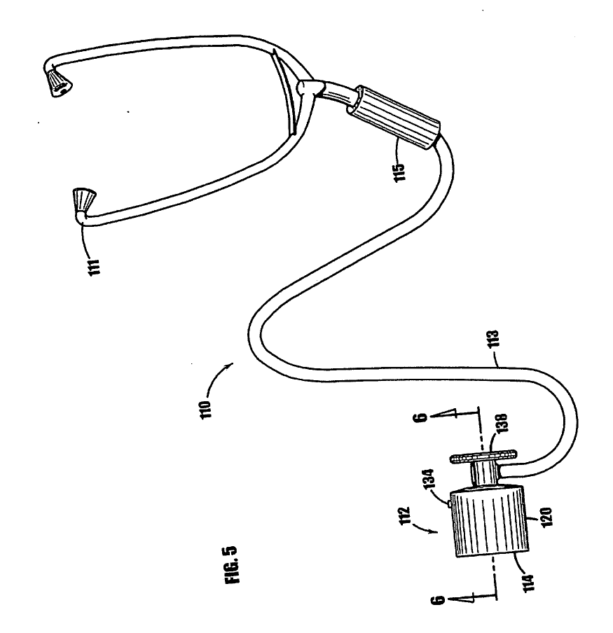

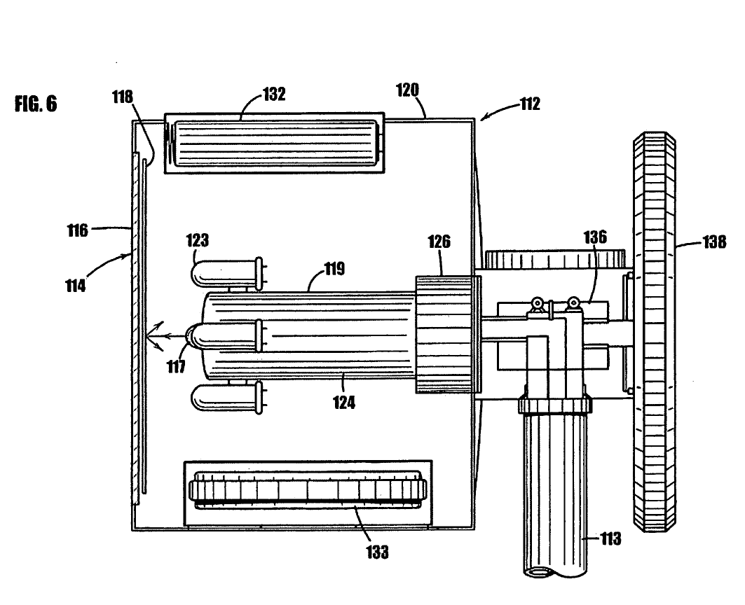

Turning to FIG. 6, the diaphragm 114 comprises a resilient layer 116 and a reflective layer or structure 118 superimposed over the resilient layer. In this embodiment of the present invention, the reflective layer 118 is made of any reflective material sold under the trademark REFLEXITE®. As may be recognized by those skilled in the pertinent art based on the teachings herein, the reflective layer 118 may be made of various other materials having comparably high coefficients of reflectivity, which are currently or later become known for performing the functions of the exemplary reflective layer or structure. A laser diode support 119 is mounted to an inner surface of the housing 120, and projects outwardly towards the diaphragm. A laser diode 117 is mounted to the free end of the support 119, and emits a beam of laser light towards the center of the reflective layer 118. A battery 133 is mounted to the housing 120, and is electrically connected to the laser diode 117 via a power switch 134 (FIG. 7) for supplying power to the diode. As may be recognized by those skilled in the pertinent art based on the teachings herein, the power switch 134 may comprise a single-position double-throw (SPDT) switch for fulfilling the function of the power switch 134. Phototransistors 123 are mounted to the laser support 119. The phototransistors 123 receive the reflected laser light directed by the reflective layer 118, and generate outputs indicative of the instantaneous position of the diaphragm 114 that are proportional to the infrasonic movements of the cardiac apical impulse.

As may be recognized by those skilled in the pertinent art based on the teachings herein, the face of the reflective layer 118 may be specially configured for directing a high percentage of the incident photons directly towards the phototransistors 123, such as, for example, by having a slightly convex shape, one or more aspheric or condensing lenses, a laser splitter mounted thereon, and/or like features for better directing the reflected light towards the phototransistors. An electronic circuit 124 is mounted to the housing 120. The electronic circuit 124 receives the outputs from the phototransistors 123, and generates signals indicative of the movement of the diaphragm 114. A speaker 126 is mounted to the housing and produces audible outputs corresponding to the signals generated and amplified by the circuit 124, and indicative of the movement of the diaphragm 114. The sensor assembly 112 further comprises an acoustic diaphragm 138 mounted to the housing 120 at the end opposite that of the diaphragm 114. A selection manifold 136 is also mounted to the housing 120, and may be acoustically coupled to either the acoustic diaphragm 138 or the speaker 126 by rotating the manifold 180 degrees relative to the housing 120.

Turning now to FIG. 7, the electronic circuit 124 is illustrated in further detail. The phototransistors 123 are connected in parallel, emitters to emitters and collectors to collectors, so that the reception of laser light at any one or more of their respective gates will generate a signal to excite the circuit. The collectors are connected to a resistor 181, which, in turn, is connected to the positive voltage potential (“V_{BAT}”) terminal of a battery 132. The negative terminal of the battery 132 is connected to ground potential. The collectors are also connected to a capacitor 182, which is then connected to a resistor 183. The resistor 183 is connected to an inverting input of an op-amp 184. An output of the op-amp 184 is connected to a feedback resistor 185, which is then connected back to the inverting input of the op-amp. The non-inverting input of the op-amp 184 is connected to a voltage drop resistor 186, which is connected in turn to V_{BAT}. The non-inverting input of the op-amp is also connected to a parallel combination of a capacitor 187 and a resistor 188, which are then connected to ground potential. The op-amp 184 functions as an amplifier for the signal generated by the phototransistors 123, which, in turn, is indicative of a cardiac motion or impulse. The output of the op-amp 184 is further connected to a capacitor 189. The capacitor 189 is connected, in turn, to a potentiometer 190. The fixed output of the potentiometer 190 is connected through a capacitor 191 to V_{BAT}. The fixed output terminal of the potentiometer 190 is further connected to an inverting input of a comparator 192. The variable output terminal of the potentiometer 190 is connected to a resistor 193, which is then connected to a non-inverting input of the comparator 192. The adjustment of the potentiometer 190 affects the duration of the pulse generated by the comparator 192. The output of the comparator 192 is connected to a capacitor 194, which is ultimately connected to a first terminal of the speaker 126. A second terminal of the speaker 126 is connected to ground potential. Thus, the speaker 126 is activated for each rise in amplitude of a cardiac apical impulse waveform detected by the phototransistors 123.

In operation, the sensor assembly 112 of the cardiac impulse detector 110 is placed on the cardiac apical impulse of a patient, with the resilient layer 116 contacting the patient's chest surface at the cardiac apical impulse point. A cardiac apical impulse induces motion of the resilient layer 116, which in turn causes motion of the diaphragm 114 and the reflective layer 118. The laser diode 117 emits laser light towards the reflective layer 118. The light is reflected by the reflective layer 118, and received by the phototransistors 123. The circuit 124 incorporates the phototransistors in an electronic motion detection scheme as described above, and drives the speaker 126 corresponding to motions of the resilient layer 116, which is hence keenly indicative of the cardiac apical motion.

The cardiac impulse detector 110 is capable of detecting and audibly representing the infrasonic vibrations of the cardiac apical impulse. There are three repetitive major vibrations detected by this device during a regular heart rhythm.

The first of these vibrations occurs at the beginning of the left ventricular (“LV”) recoil and causes the detector 110 to produce a first audible signal through the speaker 126 representing the beginning of LV recoil. The second of these vibrations occurs at the beginning of LV ejection and causes the detector 110 to produce a second audible signal through the speaker 126 representing the beginning of LV ejection, and the third of these vibrations occurs at the beginning of LV filling and causes the detector 110 to produce a third audible signal through the speaker 126 representing the beginning of LV filling. The first audible signal occurs in close timing to the normal first heart sound and the counted pulse, and the second audible signal occurs in close timing to the normal second heart sound. The third audible signal occurs after the second heart sound. These three audible signals represent directional change of the cardiac apical impulse motion.

These three audible signals are easily recognized and learned by a caregiver. Additional audible signals, which represent abnormal vibrations that are detected during ventricular filling, would thus represent gallops and indicate cardiac pathology. This embodiment also offers the advantages of electronic storage, playback, compression and analysis of signals representative of normal and abnormal infrasonic vibrations of the cardiac apical impulse. S3 and S4 types of gallops are detectable using the cardiac impulse detector 110. An extra vibration detected and audibly indicated by speaker 126 and/or visually indicated preceding the first vibration represents an S4 gallop. An extra vibration detected soon after the third vibration represents an S3 gallop. These detected vibrations and associated output signals could also be displayed graphically using analog or digital processing, electronically interfaced to a charge coupled device or LCD screen. The analog or digital processing also affords electronic storage, playback, compression and analysis of normal and abnormal output signals, indicative of normal and abnormal vibrations.

As may be recognized by those skilled in the pertinent art based on the teachings herein, numerous different processing circuits may be added or substituted for the electronic circuit 124 disclosed herein, in order to produce various signals indicative of particular types of gallops and other abnormal cardiac apical impulses. In addition, as may be recognized by those skilled in the pertinent art based on the teachings herein, when signals indicative of both the infrasonic impulses and the acoustic heart sounds are made present in electronic form, the electronic circuit may utilize the signals indicative of acoustic sounds to supplement the qualification, analysis, and/or categorization of gallops and other abnormal cardiac apical impulses.

In FIG. 8, a third embodiment of the cardiac impulse detector sensor assembly of the present invention is indicated generally by the reference numeral 212. The cardiac impulse detector sensor assembly 212 is substantially similar to the cardiac impulse detector sensor assembly 112 described above, and therefore like reference numerals preceded by the numeral 2 are used to indicate like elements. The cardiac impulse detector sensor assembly 212 of FIG. 8 differs from that of FIG. 6 primarily in that the sensor assembly 212 utilizes a convex reflector and convex collecting lenses with a compatible sensing scheme in lieu of the laser reflector of FIG. 6. The application and overall operation of the cardiac impulse detector sensor assembly 212 is substantially similar to that of the cardiac impulse detector sensor assembly 112.

As shown in FIG. 8, the cardiac impulse detector sensor assembly 212 comprises a flexible diaphragm 214 mounted about its circumference to a housing 220. An electrical power switch 234 is mounted to the housing 220 for activating the cardiac impulse detector. An audio amplifier 215 is connected to the tubing 213, and is electrically connected to the power switch 234, for enhancing the quality of the audio signal.

The diaphragm 214 is connected on its inside surface to a convex reflective structure 218. In this embodiment of the present invention, the reflective structure 218 is coated on its outer surface with any reflective material, such as that sold under the trademark REFLEXITE®. As may be recognized by those skilled in the pertinent art based on the teachings herein, the reflective structure 218 may comprise various other materials having comparably high coefficients of reflectivity, which are currently or later become known for performing the functions of the exemplary reflective surface of the structure. A laser diode support 219 is mounted to an inner surface of the housing 220, and projects outwardly towards the diaphragm 214. A laser diode 217 is mounted to the free end of the support 219, and emits a beam of laser light towards the center of the reflective structure 218. A battery 233 is mounted to the housing 220, and is electrically connected to the laser diode 217 via a power switch 234 for supplying power to the diode. Condensing lenses 221 are mounted to an inner wall of the housing 220, and are optically coupled to phototransistors 223, which are mounted to the laser support 219. The phototransistors 223 receive the reflected laser light through the condensing lenses 221 that is directed by the reflective structure 218, and generate outputs indicative of the instantaneous position of the diaphragm 214 that are proportional to the infrasonic movements of the cardiac apical impulse.

An electronic circuit 224 is mounted to the housing 220. The electronic circuit 224 receives the outputs from the phototransistors 223, and generates signals indicative of the movement of the diaphragm 214. The electronic circuit 224 is substantially similar to the electronic circuit 124, described above.

In operation, the sensor assembly 212 of the cardiac impulse detector 210 is placed on the cardiac apical impulse point of a patient, with the flexible diaphragm 214 contacting the patient's chest surface at the cardiac apical impulse point. A cardiac apical impulse induces motion of the diaphragm 214 and the reflective structure 218. The laser diode 217 emits laser light towards the reflective structure 218. The light is reflected by the reflective structure 218, condensed by the condensing lenses 221, and received by the phototransistors 223. The circuit 224 incorporates the phototransistors in an electronic motion detection scheme as described above, and drives the speaker 226 corresponding to motions of the flexible diaphragm 214, which is keenly indicative of the cardiac apical motion.

The cardiac impulse detector 210 detects and audibly represents the infrasonic vibrations of the cardiac apical impulse. There are three representative major signals detected by this device during a regular heart rhythm. The first audible signal produced by the detector through the speaker 226 represents the beginning of left ventricular recoil, the second audible signal produced by the detector represents the beginning of left ventricular ejection, and the third audible signal produced represents the beginning of left ventricular filling. These three audible signals are easily learned by a caregiver. Additional sounds produced by the detector, which represent abnormal vibrations detected during ventricular filling, would thus represent gallops and indicate cardiac pathology. As may be recognized by those skilled in the pertinent art based on the teachings herein, these signals may be displayed graphically as well as audibly, such as, for example, by using analog or digital processing, a charge coupled device and a LCD output screen. This embodiment also offers the advantages of electronic storage, playback, compression and analysis of normal and abnormal signals.

The cardiac impulse detector 210 further detects S3 and S4 gallops. When a patient presents with an S3 gallop, the speaker 226 will produce a sound immediately following the third sound. When a patient presents with an S4 gallop, the speaker 226 will produce a sound immediately preceding the first sound.

As may be recognized by those skilled in the pertinent art based on the teachings herein, numerous different processing circuits may be added or substituted for the electronic circuit 224 disclosed herein, in order to produce various signals indicative of particular types of gallops and other abnormal cardiac apical impulses. In addition, as may be recognized by those skilled in the pertinent art based on the teachings herein, when signals indicative of both the infrasonic impulses and the acoustic heart sounds are made present in electronic form, the electronic circuit may utilize the signals indicative of acoustic sounds to supplement the qualification, analysis, and/or categorization of gallops and other abnormal cardiac apical impulses.

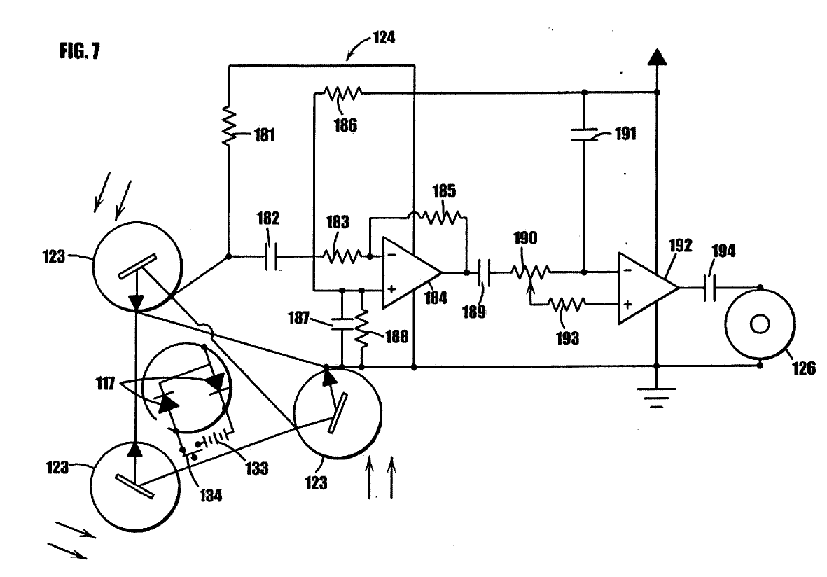

Referring to FIG. 9, a fourth embodiment of the cardiac impulse detector of the present invention is indicated by reference numeral 310. The cardiac impulse detector sensor 310 is substantially similar to the cardiac impulse detector identified and discussed above with respect to the various embodiments of the present invention, and therefore like reference numerals proceeded by the numeral 3, or preceded by the numeral “3” instead of the numerals “2” or “1”, are used to indicate like elements. In this embodiment, the cardiac impulse detector 310 preferably utilizes an appropriate light source and compatible sensing scheme. The cardiac impulse detector 310 preferably comprises at least one sensor assembly 312 and at least one flexible substrate in the form of an adhesive reflective patch (ARP) 314. As shown, in a preferred aspect of the invention, the ARP 314 is remote from the sensor assembly 312 and preferably positioned over the skin surface of the chest wall at least substantially adjacent the left ventricle (LV) apex area of the heart, such as at the cardiac apical impulse point or other desired predetermined locations as described further below. The term “patch” is used herein to mean a substrate or body that may be affixed to the skin at an impulse point or other desired location to enable a sensor to detect impulses, such as apical impulses. As may be recognized by those of ordinary skill in the pertinent art based on the teachings herein, the flexible substrate may take any of numerous different shapes, configurations and/or forms that are currently or later become known.

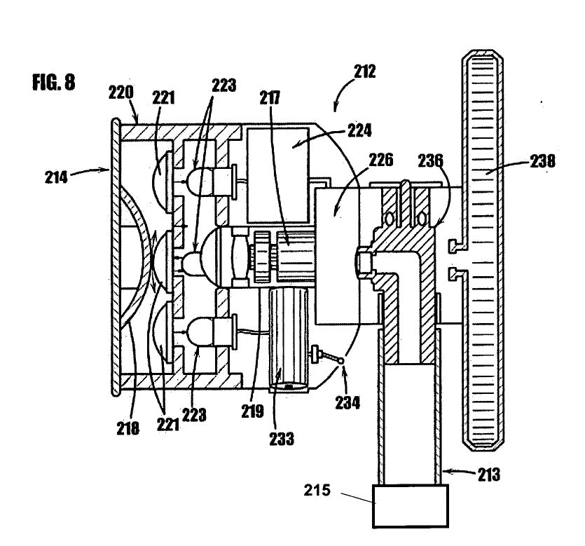

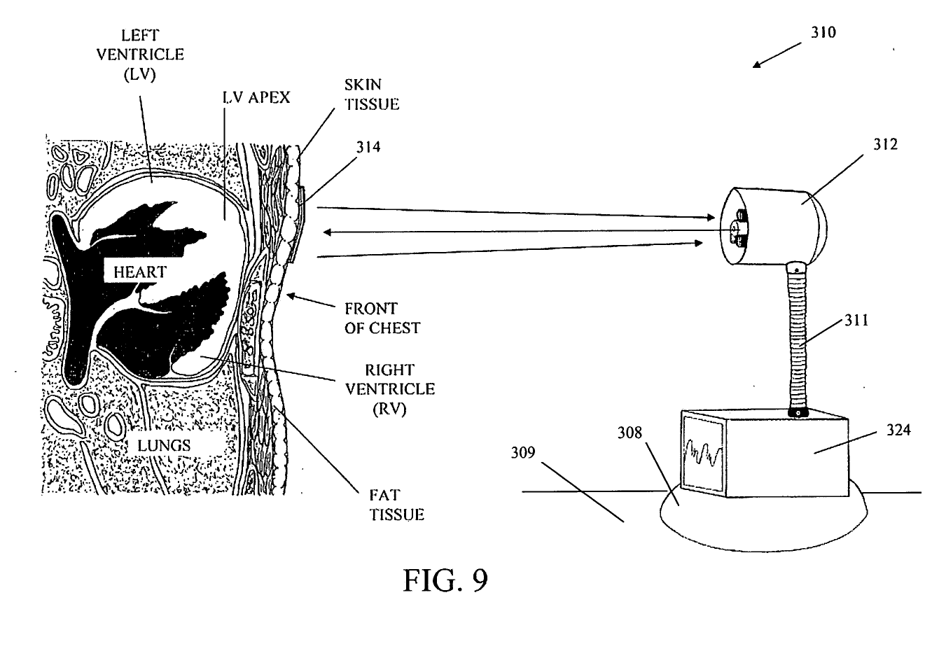

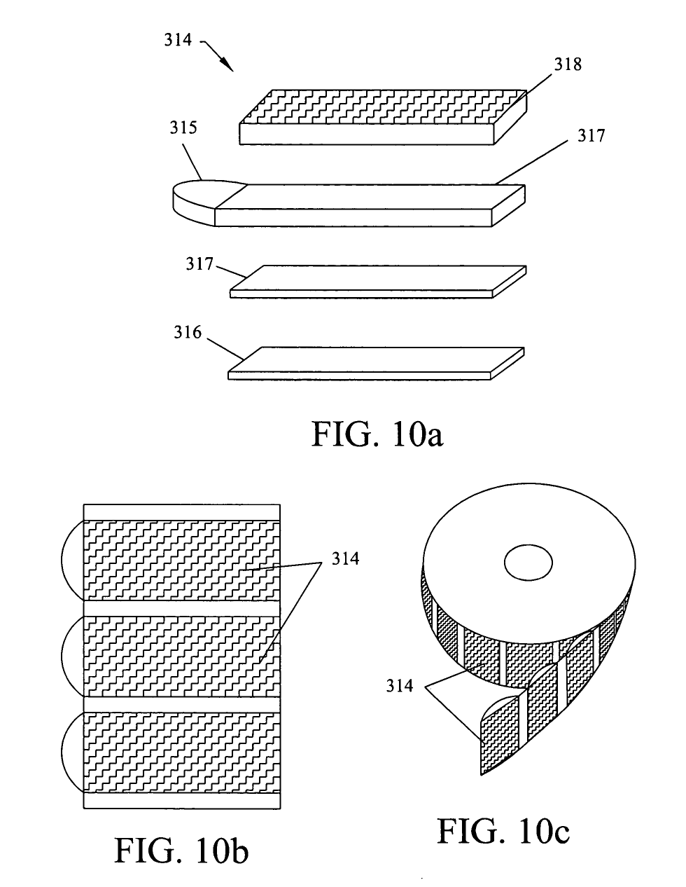

Referring to FIG. 10, the ARP 314 essentially has at least one adhesive skin contacting (ASC) layer 316 and at least one reflective layer 318. In addition, the ARP 314 may have one or more intermediate layers 315 separating the at least one ASC layer 316 and the at least one reflective layer 318. In one aspect of the present invention, at least one of the intermediate layers 315 may be provided with a tab 315a for convenient handling of the ARP 314. The tab 315a may form an integral part of one or more of the intermediate layers 315 or may be separably connected therewith via any known method for accomplishing such an operation. The ASC layer 316 is preferably a pressure-sensitive adhesive layer of a type known to those of ordinary skill in the pertinent art. In addition, the ARP 314 may be provided on a releasable backing contacting the ASC layer 316, or plural ARPs 314 may be mounted on a common releasable backing in a manner known to those of ordinary skill in the pertinent art. Preferably, the peripheral shape of the reflective layer 318 and thus of the ARP 314 substantially matches the peripheral shape of the light beam transmitted thereon. However, as may be recognized by those of ordinary skill in the pertinent art based on the teachings herein, the reflective layer and/or ARP may take any of numerous different peripheral shapes or configurations that are currently known or later become known. For example, the reflective layer and/or ARP may utilize a metallized UV stabilized polymeric film and a pressure sensitive adhesive backing. Alternatively, the reflective layer and/or ARP may utilize a non-metallized acrylic film sealed to a vinyl carrier and an acrylic based adhesive backing. The type of reflective or retro-flective material can significantly improve the efficiency associated with the reflective layer and/or the ARP. For instance, a material incorporating a cube corner prismatic system, which system utilizes the various prism facets to reflect light back toward a light source is three times more efficient than a material incorporating a spherical lens glass bead system, which system focuses light on a rear surface of the bead and reflects it back toward the light source. Thus, at least in one aspect of the present invention, the reflective layer and/or ARP preferably utilize a resilient high performance retro-flective material having in the range of about 47,000 reflective prisms per square inch, for example.

The ARP thus defines a flexible substrate that is resilient and capable of intimately contacting the skin surface region of interest for movement therewith to, in turn, enable optical detection of, for example, infrasonic cardiac apical impulses. In a currently preferred embodiment of the present invention, the ARPs are single use, disposable items. In one such embodiment, plural ARPs may be provided on a common releasable backing. The releasable backing may be provided in the form of a flat sheet as shown in FIG. 10b, or is provided in the form of a roll as shown in FIG. 10c, wherein plural ARPs are mounted on either the flat sheet or roll and are successively peeled therefrom as needed by a practitioner.

As with each of the previously discussed embodiments of the present invention, in this embodiment the reflective layer 318 may be made of any of a variety of reflective materials, including for example, the reflective material sold under the trademark REFLEXITE®. As may be recognized by those skilled in the pertinent art based on the teachings herein, suitable reflective materials having coefficients of reflectivity that are comparable to that of REFLEXITE®, which are currently known or may later become known, and which are suitable to accomplish the purposes of the exemplary reflective layer of the present invention also may be used.

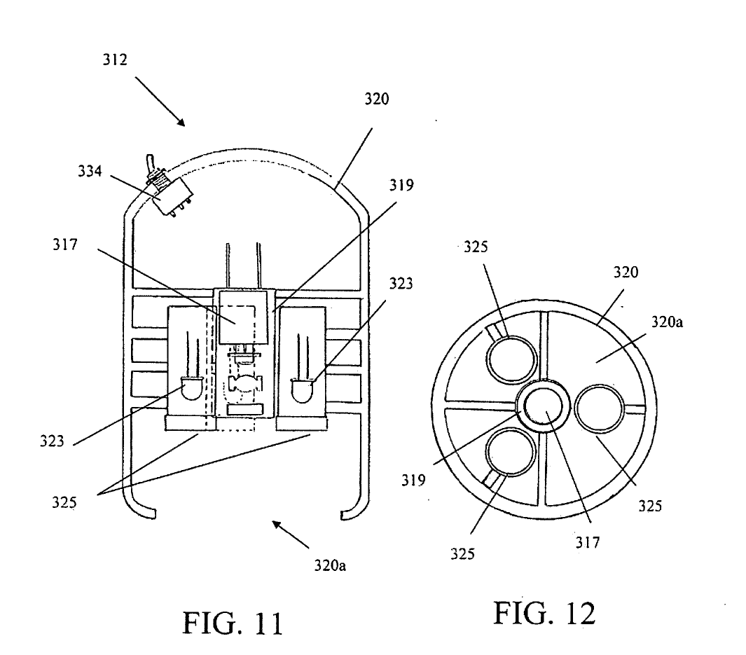

Referring to FIGS. 11 and 12, the sensor assembly 312 preferably has a support 319 for holding at least one light source 317. The support 319 is preferably positioned in a housing 320 so that the light source 317 may radiate light or other desired radiation through an opening 320a in the housing 320 and onto the ARP 314, which is preferably remotely located relative to the sensor assembly 312. A control switch 334 for supplying power to the light source 317 and/or the sensor assembly 312 as a whole also may be provided as part of the sensor assembly 312. Preferably, the sensor assembly 312 also has one or more phototransistors 323 or other optical sensors preferably supported by the support 319. The phototransistors 323 are preferably suitable to receive light modulated by the reflective layer 318 of the ARP 314, and to generate outputs indicative of the instantaneous position of the ARP 314 that are proportional to the infrasonic movements of a cardiac apical impulse.

Referring again to FIG. 9, it is noted that the cardiac impulse detector sensor 310, in one aspect of the present invention, is operatively connected to a base structure 308 suitable to fixedly support the sensor assembly 312 and/or the light source 317 with respect to a support surface 309. In addition, the cardiac impulse detector sensor 310 can have an adjustable arm structure 311 suitable for controlling, directing, focusing and/or otherwise manipulating the orientation of the light source 317 with respect to the ARP 314 so that light from the light source 317 may be adjustably directed or focused as desired onto the ARP 314. Thus, the base structure 308 and the arm structure 311 allow for optimally directing or focusing light from light source 317 onto the ARP 314 and maintaining the desired directed or focused position for a desired amount of time. Accordingly, the base structure 308 and the arm structure 311 advantageously facilitate in reducing unwanted signal noise.

As may be recognized by those skilled in the pertinent art based on the teachings herein, the surface of the reflective layer 318 may be specially configured for directing the highest percentage of the incident photons directly towards the phototransistors 323, such as, for example, by having a convex shape or pattern design to alter polarity. In addition or alternatively, one or more aspheric or condensing lenses or band pass filters 325, or laser splitters, or like features, may be in association with the phototransistors 323 for better directing the reflected light towards the phototransistors 323. Further, an electronic circuit 324 may be operatively connected to the sensor assembly 312 to filter and/or amplify the outputs of the phototransistors 323 and/or generate signals indicative of the movement associated with the ARP 314.

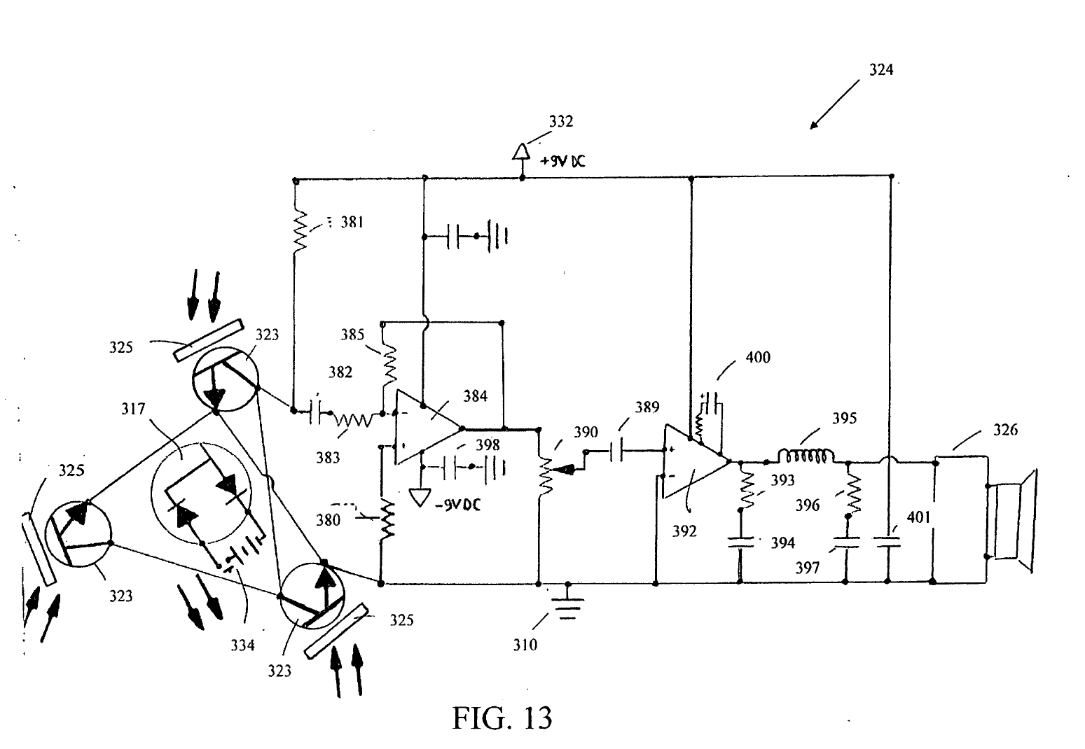

Referring to FIG. 13, the electronic circuit 324 is shown in more detail. As shown, the phototransistors 323 are connected in parallel, emitters to emitters and collectors to collectors, so that the reception of coherent light at any one or more of their respective gates will generate a signal to excite the circuit. Preferably, the emitters and/or collectors are connected to a resistor 381, which in turn, is connected to the positive voltage potential (+9VDC) terminal of dual—polarity power supply of a battery 332, for example. The ground terminal of the battery 332 is connected to ground potential and the negative terminal of the battery 332 is connected to the negative voltage potential (−9VDC).

The emitters and/or collectors also are connected to a capacitor 382, which is in turn connected to a resister 383. The resister 383 is connected to an inverting input of an Op-Amp 384 and an output of the Op-Amp 384 is connected to a feedback resister 385, which is turn connected back to the inverting input of the Op-Amp 384 to preferably increase the signal. The non-inverting input of the Op-Amp 384 is connected to a voltage drop resistor 380, which in turn is connected to ground potential. The Op-Amp 384 is further connected to ground potential. A bypass capacitor 398 connects the circuit to ground potential to prevent oscillations. The Op-Amp 384 is still further connected to a positive voltage potential and a bypass capacitor 399 connects the circuit to ground potential to prevent oscillations. The Op-Amp 384 preferably functions as an amplifier for the signal generated by the phototransistors 323, which, in turn, may be indicative of a cardiac motion or impulse. The output of the Op-Amp 384 is further connected to a potentiometer 390. The potentiometer 390 is in turn connected to a capacitor 389. The fixed output terminal of the capacitor 389 is further connected to an inverting input of a comparator 392. The non-inverting input of the comparator 392 is connected by way of a resistor 393 to a capacitor 394, which is connected to the ground potential. A second terminal of the output of the comparator 392 is connected to an inductor 395, which by way of a resistor 396 and a capacitor 397 is connected to the ground potential to act as a LC low pass filter. The second terminal of the inductor 395 is connected to an amplifier/speaker 326, which is connected to ground potential. A resistor/capacitor 400, coupled between at least two pins of the comparator 392, serves to increase the gain. A capacitor 401 preferably connects the positive voltage terminal ground potential to thereby reduce feedback. Thus, the amplifier/speaker 326 may be activated for each rise in amplitude of a cardiac apical impulse detected by the phototransistors 323.

In operation, the sensor assembly 312 of the cardiac impulse detector 310 is directed toward the ARP 314 which is in turn positioned in intimate contact with the patient's chest surface at or about the cardiac apical impulse region. A cardiac apical impulse induces motion of the resilient layer 316, which in turn causes motion of the ARP 314. The light source 317 emits light toward the ARP 314 so that such emitted light is retro-reflected by the ARP 314 and received by the phototransistors 323. The circuit 324 preferably incorporates the phototransistors 323 in an electronic motion detector scheme such as described above, and is suitable to drive the amplifier/speaker 326 and/or visual output device, such a CRT, flat panel or other type of display, corresponding to the motions of the ARP 314, which is indicative of the cardiac apical motion.

The cardiac impulse detector 310 is preferably capable of detecting and audibly and/or visually representing the infrasonic vibration of the cardiac apical impulse. There are at least four repetitive major subsonic vibrations detected by the cardiac impulse detector 310 during regular heart rhythm. The first of these subsonic vibrations occurs at the beginning of left ventricular (LV) systole and preferably causes the cardiac impulse detector 310 to produce a first signal, such as, for example, an audible signal through the speaker 126 and/or visual signal representing the beginning of LV systole. The second of these subsonic vibrations occurs at the beginning of LV diastole. Here, the LV generates suction and preferably causes the cardiac impulse detector 310 to produce a second signal representing LV recoil or the event that initiates LV filling. The third of these subsonic vibrations occurs shortly after the second, and preferably causes the cardiac impulse detector 310 to produce a third signal representing LV filling. A fourth subsonic vibration occurs right before the first subsonic vibration and causes the cardiac impulse detector 310 to produce a fourth signal representing left atrial contraction at the end of LV diastole.

The first signal occurs in close timing to the normal first heart sound and the counted pulse, and the second signal typically occurs in close timing to the normal second heart sound. The third signal typically occurs after the second heart sound, and the fourth signal typically occurs right before the first heart sound. These four signals represent outward directed changes of the cardiac apical impulse motion. Each of the four signals may be seen when such signals are electronically displayed as, for example, in FIG. 14 via reference numerals S1, S2, S3 and S4, respectively.

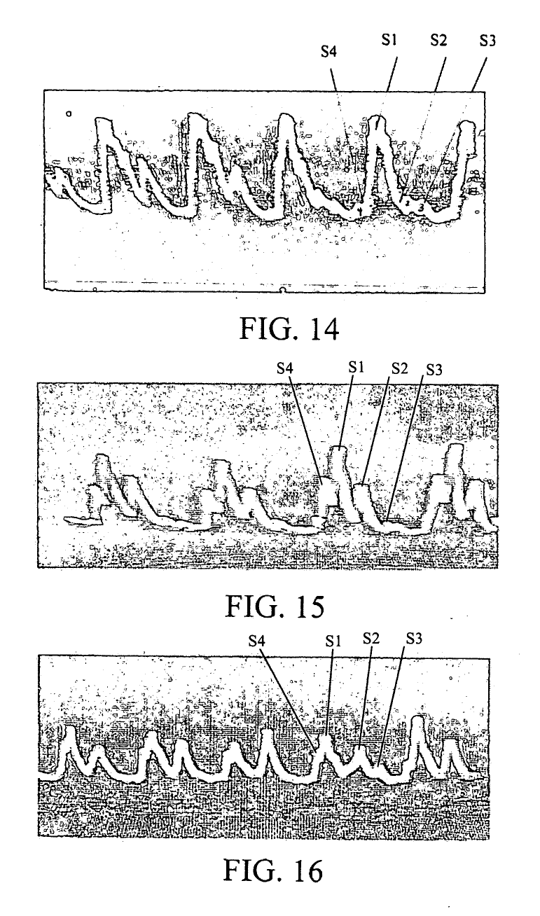

Cardiac pathology may be recognized when these signals are altered in shape, timing and/or presence. For instance, FIG. 14 is an exemplary visual representation of a signal for a normal heart. FIG. 15 is an exemplary visual representation of a signal for a hypertrophy. FIG. 16, on the other hand, is an exemplary visual representation of a signal for a myocardial infarction. As shown typically in FIGS. 14 and 15, and in contrast to FIG. 14, abnormal cardiac vibrations, such as gallops, may be recognized by the exaggeration of the configuration of the third and fourth subsonic vibrations (S3 and S4 gallops, respectfully). Congestive heart failure is recognized by the abnormal alteration of the first subsonic vibration S1 defining a relatively slow or blunted upstroke in its displayed configuration. The second subsonic vibration S2 may be delayed and altered, as shown typically in FIGS. 15 and 16, and in contrast to FIG. 14, thus further denoting cardiac pathology.

In an aspect of the present invention, cardiac pathologies observed via the cardiac impulse detector 310 may be recorded and/or stored in a database for subsequent reference and/or analysis by any suitable means for accomplishing such an operation. The observed cardiac pathologies may be automatically stored as graphical representations and/or data points. The graphical representations and/or data points are preferably suitable to allow an observed cardiac pathology to be compared with representations/data associated with normal heart function. In other aspects of the present invention, signals indicative of any difference and/or abnormality detected in an observed cardiac pathology with respect to a normal heart function may be generated and/or displayed for diagnosis. Further, an observed cardiac pathology may be automatically diagnosed as one or more abnormal pathologies by comparing the representations/data of the observed pathology to the representations/data associated with known abnormal pathologies to determine if any correlation exists. Thus, more effective and efficient diagnosis may be provided by the features of the present invention.

As may be recognized by those skilled in the pertinent art based on the teachings herein, these detected vibrations and associated output signals may be indicated audibly by means of the amplifier/speaker 326 and/or visually by means of an LED or the like, and/or displayed graphically using analog or digital processing electronically interfaced to a charge-coupled device, LCD screen or the like. The analog or digital processing preferably also affords electronic storage, playback, compression and template analysis of output signals indicative of normal and abnormal vibrations of the cardiac apical impulse.

As also may be recognized by those skilled in the pertinent art based on the teachings herein, numerous different processing circuits may be added or substituted for the electronic circuit 324 disclosed herein, in order to produce various signals indicative of particular types of gallops and other abnormal cardiac apical impulses. In addition, as may be recognized by those skilled in the pertinent art based on the teachings herein, when signals indicative of both the infrasonic impulses and the acoustic heart sounds are made present in electronic form, an electronic circuit current may utilize the signals of acoustic sounds to supplement the qualification, analysis, and/or categorization of gallops and other abnormal cardiac apical impulses.

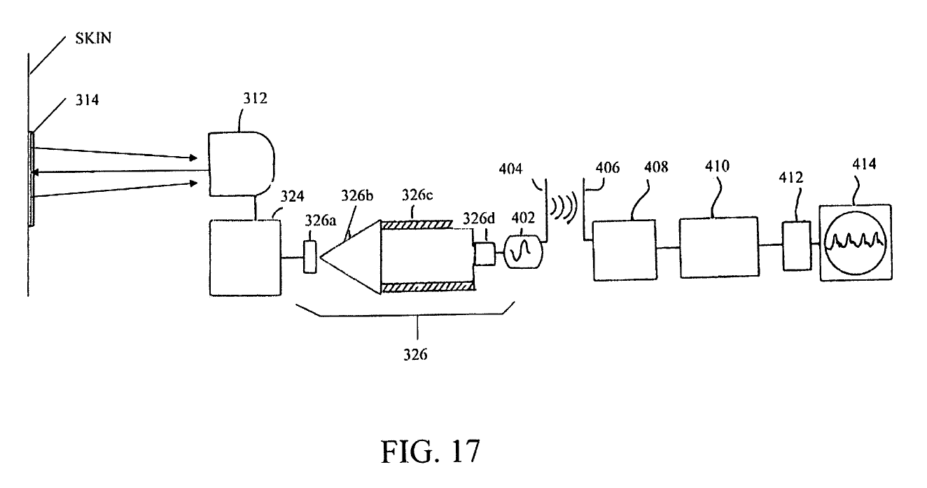

Referring to FIG. 17, in operation, the present embodiment of the cardiac impulse detector 310 may comprise the sensor assembly 312, which is preferably fixed and oriented to emit light in the direction of the ARP 314. The ARP 314 is preferably configured and/or appropriately positioned so that the retro-flected light stemming therefrom, which light may be modulated as desired via methods known in the pertinent art, is directed toward and sensed by the phototransistors 323 of the sensor assembly 312. The cardiac impulse detector 310 preferably also has at least one circuit arrangement, such as, for example, circuit 324 that is operative to amplify and filter the resultant outputs of the phototransistors 323 to generate signals indicative of the movement of the ARP 314 placed in intimate contact with a person's skin over the subsonic cardiac apical impulse point or other skin surface region of interest. The electrical signals may be converted into sound waves by way of the amplifier/speaker 326, which may include at least one amplifier 326a, at least one speaker 326b, and at least one baffled sound tunnel 326c for directing sound waves to at least one microphone 326d. A radio frequency (RF) oscillator 402 is used, for example, to broadcast broadband FM radio waves 405 by way of antennae 404, 406 to a receiver 408. This arrangement preferably acts as a filter to filter out undesirable signal noise. The amplifier 326a preferably enhances the signal, the speaker 326b and baffled sound tunnel 326c preferably concentrate the enhanced signal, and the microphone 326d, the oscillator 402, and the antennae 404, 406 efficiently forward the concentrated enhanced signal so that the receiver 408 receives a substantially clean signal. The receiver 408 in turn directs electrical signals into a sound amplitude analyzer 410, which by way of a controller 412 drives a graphic LCD display module 414 that displays the resultant modulated signal representing the subsonic cardiac apical impulse.

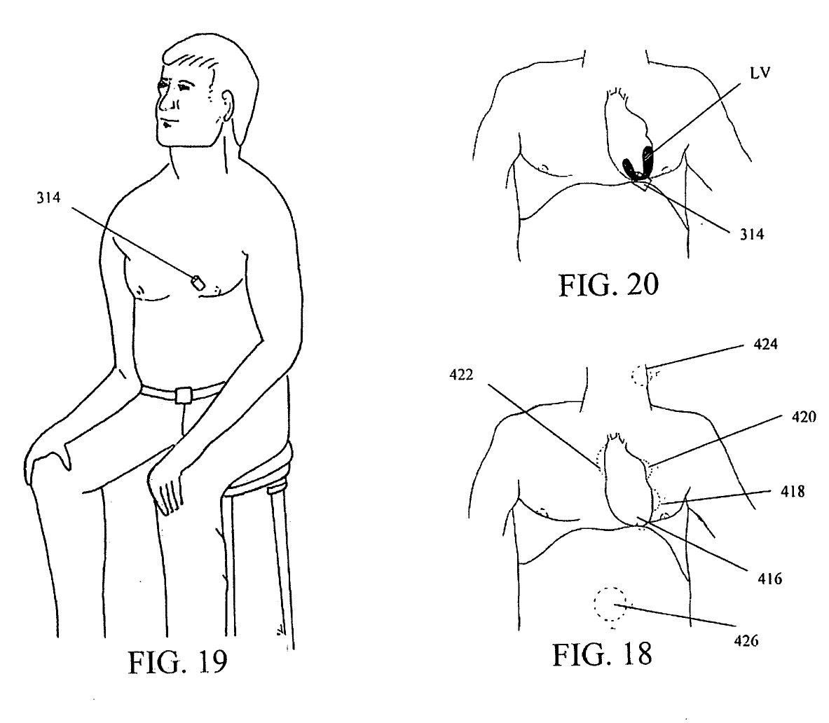

Referring to FIGS. 18 to 20, in accordance with a preferred use of the present invention, to obtain optimal results it is preferable that the ARP 314 be placed in direct contact with a person's skin at a point of maximum impulse. As shown in FIG. 18, possible points of maximum impulse may include, but are not limited to, an LV apex region 416, an LV wall region 418, a pulmonary artery dilatation region 420, an ascending aortic artery dilatation region 422, a carotid artery pulsation region 424, and/or an abdominal aortic artery pulsation region 426. Further, it is also preferable for favorable results that a person be placed in an upright sitting position such as shown in FIG. 19 with his/her chin at least slightly raised. This positioning of the person preferably causes the LV, highlighted in FIG. 20, to be brought into close contact with the chest wall and thus into closer proximity with the ARP 314. In addition, it is more preferable for optimal results that the person's legs be raised and/or positioned in horizontal manner. This raising/positioning of the person's legs preferably causes blood to flow out of the legs and into the heart and results in the heart expanding and/or moving at least into closer proximity with the chest wall and/or preferably into contact with the apical impulse point or area thereof.

One advantage of the above-described embodiments of the present invention is that an audible and/or visual signal indicative of an infrasonic cardiac apical impulse may be generated for contemporaneous diagnosis by a medical caregiver.

Another advantage of the above-described embodiments of the present invention is that an audible and/or visual signal indicative of an S3 or S4 gallop may be generated during a brief physical examination of a patient.

A further advantage of the above-described embodiments of the present invention is that an audible and/or visual signal is indicative of an infrasonic cardiac apical impulse may be supplied in context with traditional acoustic cardiac sounds to promote efficient examination and diagnosis of a patient.

An additional advantage of the above-described embodiments of the present invention is that an audible and/or visual signal is indicative of an infrasonic cardiac apical impulse may be generated in a medical school curriculum to promote enhanced understanding of the clinical manifestations of various heart diseases.

Another advantage of the above-described embodiments of the present invention is that a patient may be located at a distance from the sensor assembly, thereby allowing the sensor assembly to be fixedly positioned relative to the patient resulting in more accurate measurement of the cardiac apical impulse.

Still another advantage provided by the above-described embodiments of the present invention is that the reflective patches may be disposable and easily adjusted as desired to provide optimal results.

Yet another advantage provided by the above-described embodiments of the present invention is that the cardiac apical impulses may be visually displayed for efficient and convenient analysis or study.

Yet still another advantage provided by the above-described embodiments of the present invention is that the visual data representing a particular cardiac apical impulse may be manually or automatically stored, compared and/or analyzed against other cardiac apical impulse data in an efficient and effective manner via graphical representations and/or data points that allow observed cardiac apical impulse data to be compared and/or analyzed with the cardiac apical impulse data associated with normal heart function and/or abnormal cardiac pathologies.

As may be recognized by those skilled in the pertinent art based on the teachings herein, numerous changes may be made to the above described and other embodiments of the present invention without departing from its scope or spirit as defined in the appended claims. For example, alternate or supplemental sensors capable of sensing the low frequency vibrations or impulses generated by the heart include piezoelectric crystals, piezoelectric films, accelerometers, silicon pressure transducers, lasers, and other displacement devices. These low frequency vibrations also can be detected by electromagnetic field devices such as inductance transducers. Therefore, any of a number of sensing devices presently available or later developed may be used to augment or replace the sensors used for exemplary purposes herein.

Similarly, the particular hardware used for the acoustic diaphragm may be electronically augmented, and the earpiece, tubing, and housing may be replaced with hardware having similar functionality without departing from the scope and spirit of the present invention.

Likewise, the acoustic diaphragm itself may be replaced with a microphone, piezoelectric audio sensor, or similar mechanism, such that the processing of the infrasonic and audible cardiac motions may be done electronically to produce an audible output for the first time at one or more earpiece transducers.

In addition, a single sensor may be used to sense both infrasonic motions and audible sounds. For example, a piezoelectric sensing diaphragm may be used to sense both infrasonic motions and audible sounds when combined with a sensing protuberance capable of transmitting infrasonic motions and audible frequencies such that the audible sounds are not damped out by the mechanical loading of the sensing protuberance against the tissue of a patient.

Accordingly, this Detailed Description of the Preferred Embodiments is to be taken in an illustrative as opposed to a limiting sense.

What is claimed is:

1. A flexible substrate engageable with a patient's skin for detecting infrasonic cardiac apical impulses, the flexible substrate being operable in connection with an apparatus including a light source spaced apart from and facing the substrate to transmit light thereon, an optical sensor spaced apart from and facing the substrate for receiving reflected light from the substrate and generating a signal indicative of movement of the substrate and corresponding to a subaudible motion of the contacted skin at an infrasonic cardiac apical impulse point, and a circuit coupled to the optical sensor for generating at least one of (i) an audible output and (ii) a visual output, in response to the signal and indicative of an infrasonic cardiac apical impulse, the flexible substrate comprising:
(i) a flexible skin-contacting surface located on one side of the substrate that is disposable in contact with a skin surface region of a patient defining an infrasonic cardiac apical impulse point, and is movable with the contacted skin surface region in response to a subaudible motion of the contacted skin at the infrasonic cardiac apical impulse point, and (ii) a flexible reflective surface located on an opposite side of the substrate relative to the skin-contacting surface and movable with the skin-contacting surface in response to a subaudible motion of the contacted skin at the infrasonic cardiac apical impulse point for reflecting light transmitted thereon by the light source onto the optical sensor.

2. A flexible substrate as defined in claim 1, further comprising an adhesive layer defining at least a portion of the skin-contacting surface for releasably engaging the patient's skin.

3. A flexible substrate as defined in claim 2, further comprising means for at least one of grasping and handling the flexible substrate.

4. A flexible substrate as defined in claim 3, wherein said means for at least one of grasping and handling the flexible substrate is a tab operatively connected to at least one of the skin-contacting surface and the reflective surface.

5. A flexible substrate as defined in claim 1, further comprising at least one intermediate layer separating said skin-contacting surface and said reflective surface.

6. A flexible substrate as defined in claim 4, wherein said at least one intermediate layer has a tab suitable to facilitate at least one of grasping and handling of the flexible substrate.

7. A cardiac impulse detecting system comprising:
at least one reflective patch;
at least one sensor assembly; and
a circuit assembly for detecting, audibly and/or visually representing, and/or quantifying infrasonic vibrations associated with a cardiac apical impulse.

8. The system of claim 7, wherein said reflective patch is remote from said at least one sensor assembly and/or said circuit assembly.

9. The system of claim 8, wherein said at least one sensor assembly is fixedly positioned relative to said at least one reflective patch.

10. The system of claim 7, wherein said reflective patch has at least one adhesive layer and at least one reflective layer.

11. The system of claim 10, wherein said reflective patch has a means for grasping and/or handling associated therewith.

12. The system of claim 7, wherein said at least one sensor assembly has at least one light source for emitting light in a desired direction.

13. The system of claim 12, wherein said at least one sensor assembly has one or more light sensors for detecting reflected light.

14. The system of claim 13, wherein said at least one light source and said one or more light sensors are operatively connected to said circuit assembly.

15. The system of claim 14, wherein said circuit assembly is operatively connected to an amplifier/speaker assembly.

16. The system of claim 15, wherein said amplifier/speaker assembly is operatively connected to an oscillator for broadcasting radio signals into a receiver for directing electrical signals to an amplitude analyzer, which by way of a controller may drive a graphic display module suitable for visually demonstrating a subsonic cardiac apical impulse.

17. A method for detecting a cardiac pathology comprising the steps of;
providing a cardiac impulse detector having at least one sensor assembly with means for emitting and/or detecting light, and means suitable for manipulating data from said sensing assembly for generating signals indicative of the movement at least one remote reflective patch;
sitting a person at a distance from said sensing assembly in an upright position;
placing said at least one reflective patch in intimate contact with the person's skin at a cardiac apical impulse point;
orienting said at least one sensor assembly to respectively emit and detect light in and from the direction of said at least one reflective patch; and
generating signals indicative of the movement of the at least one reflective patch in response to a cardiac apical impulse.

18. The method of claim 17, further comprising the step of representing said indicative signals via a visual display.

19. The method of claim 17, further comprising the step of representing said indicative signals via an audible indicator.

20. A flexible substrate engageable with a patient's skin for detecting infrasonic cardiac apical impulses, the flexible substrate being operable in connection with an apparatus including means spaced apart from and facing the substrate for transmitting light thereon, means spaced apart from and facing the substrate for receiving reflected light from the substrate and generating a signal indicative of movement of the substrate and corresponding to a subaudible motion of the contacted skin at an infrasonic cardiac apical impulse point, and means for generating at least one of (i) an audible output and (ii) a visual output in response to the signal and indicative of an infrasonic cardiac apical impulse, the flexible substrate comprising:
(i) first means located on one side of the substrate for flexibly contacting a skin surface region of a patient defining an infrasonic cardiac apical impulse point, and moving with the contacted skin surface region in response to a subaudible motion of the contacted skin at the infrasonic cardiac apical impulse point, and (ii) second means located on an opposite side of the substrate relative to the first means and movable with the skin-contacting surface in response to a subaudible motion of the contacted skin at the infrasonic cardiac apical impulse point for reflecting light transmitted thereon by the means for transmitting onto the means for receiving reflected light.

21. A flexible substrate as defined in claim 19, further comprising third means for releasably engaging the patient's skin.

22. A flexible substrate as defined in claim 19, further comprising means for gripping the flexible substrate.

23. A flexible substrate as defined in claim 21, wherein the means for gripping is a tab.

24. A flexible substrate as defined in claim 19, wherein the first means is a skin-contacting surface.

25. A flexible substrate as defined in claim 19, wherein the second means is a reflective surface.

26. A flexible substrate as defined in claim 20, wherein in the means for releasably engaging is a pressure-sensitive adhesive backing.
